(19) United States
(12) Patent Application Publication
(10) Pub. No.: US 2008/0206177 A1
(43) Pub. Date: Aug. 28, 2008

(54) Cosmetic composition comprising at least one cationic polyurethane and at least one polyethylene glycol ester, to be applied during hair dressing

(76) Inventor: Pascale Cothias, Montigny le Bretonneux (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/007,625

(22) Filed: Jan. 14, 2008

Related U.S. Application Data
(60) Provisional application No. 60/903,302, filed on Feb. 26, 2007.

(30) Foreign Application Priority Data
Jan. 12, 2007 (FR) 0752653

(51) Int. Cl. A61K 8/72; A61K 47/30; A61Q 5/06

(52) U.S. Cl. 424/70.11; 514/772.4

(57) ABSTRACT

The present disclosure relates to a cosmetic composition comprising in a cosmetically acceptable medium:
(i) at least one cationic polyurethane comprising at least one non-ionic unit derived from at least one polymer chosen from olefinic homopolymers and copolymers, and
(ii) at least one polyethylene glycol ester.

The present disclosure relates to a method for treating hair using the compositions herein defined.

This application claims benefit of U.S. Provisional Application No. 60/903,302, filed Feb. 26, 2007, the contents of which are incorporated herein by reference. This application also claims benefit of priority under 35 U.S.C. § 119 to French Patent Application No. FR 0752653, filed Jan. 12, 2007, the contents of which are also incorporated herein by reference.

The present disclosure relates to new cosmetic compositions comprising the combination of at least one cationic polyurethane comprising non-ionic units derived from at least one olefinic homopolymer and/or copolymer, and at least one polyethylene glycol ester.

Extensive research has been conducted on cosmetics with respect to the formation of deposits and films possessing elastic properties. Most parts of the human body that are potential recipients of such cosmetic deposits, such as skin, lips, hair, eyelashes, and nails, are subject to substantial deformations and mechanical stresses. Most cosmetic films and deposits should be able to resist such stresses and follow such deformations without breaking.

The use of polyurethanes in cosmetics is known and is described for example in patents WO 94/13724 and EP 0 619 111.

The polyurethanes described in those documents form brittle films that are not acceptable for cosmetic application.

Other physiologically acceptable polymers exist, such as acrylic polymers, but such polymers generally form highly sticky deposits, which is a drawback for most cosmetic applications.

The use of elastic cationic polyurethanes in cosmetic compositions, for example in styling compositions, is known.

French patent application FR 2 815 350 describes elastic cationic polyurethanes and their use for formulating hair sprays and hair styling compositions to enhance hair suppleness. Compositions disclosed therein provide a more natural way to hold hair styles as compared to that obtained with usual fixing polymers.

French patent application FR 2 833 960 describes cosmetic styling compositions and rinse-off hair styling compositions, for example styling shampoos, comprising a self-adhering cationic or amphoteric polyurethane.

The inventor has discovered, surprisingly, that using at least one cationic polyurethane comprising units derived from an olefinic homopolymer and/or copolymer in a hair styling composition can provide a desirable hold when styling hair in the absence of mechanical stresses, water, and moisture. However, the presence of such stimuli can lead to deterioration of the desirable hold.

The inventor has further surprisingly discovered that cosmetic compositions comprising at least one cationic polyurethane comprising units derived from an olefinic homopolymer and/or copolymer as well as at least one polyalkylene glycol ester can enable one to obtain hair styles that can better resist deterioration in the presence of mechanical stresses, water, and moisture, than the hereabove described formulations do. These cosmetic composition may also enable the hair hold to look more natural.

Accordingly, one aspect of the present disclosure relates to a cosmetic composition, comprising, in a cosmetically acceptable aqueous medium:

(i) at least one cationic polyurethane comprising at least one non-ionic unit derived from an olefinic homopolymer and/or copolymer, and

(ii) at least one polyethylene glycol ester.

In at least one embodiment the at least one cationic polyurethane comprises:

(a) cationic units (a) resulting from the reaction of at least one amine chosen from tertiary and quaternary amines comprising at least two labile hydrogen-containing reactive functional groups,

(b) non-ionic units (b) derived from non-ionic polymers possessing labile hydrogen-containing reactive functional groups at their ends, wherein at least one of the (b) units, for example, at least 50% by weight of the (b) units, such as all of the (b) units are present as at least one non-ionic unit (b1) which results from the reaction of at least one polymer chosen from olefinic homopolymers and/or copolymers possessing labile hydrogen-containing reactive functional groups at their ends, and

(c) units derived from at least one diisocyanate.

As used herein, a “cationic unit” means any unit that, either due to its own chemical nature, or because of its environment and/or the pH value of the composition which comprises it is in a cationic form.

As used herein, “labile hydrogen-containing reactive functional groups” mean functional groups that are able, after the removal of a hydrogen atom, to form covalent bonds with another compound, for example, the isocyanate functional groups of the compounds forming the (c) units. Non-limiting examples of such functional groups include hydroxyl, primary amine (—NH₂) or secondary amine (—NHR), or thiol (—SH) groups.

Polycondensation of compounds possessing these labile hydrogen-containing reactive functional groups with diisocyanates results in polyurethanes, polyureas, or polythiourethanes, depending on the nature of the labile hydrogen-containing reactive functional groups (—OH, —NH₂ and —NHR, or —SH, respectively). Despite this standard nomenclature, these polymers, e.g., polyurethanes, polyureas, and polythiourethanes, are generally referred to as “polyurethane,” in the present disclosure for simplification purposes. In at least one embodiment of the present disclosure, the polymers can be, for example, authentic polyurethanes, i.e., the hydrogen-containing reactive functional group is an —OH.

When the tertiary or quaternary amines forming the cationic units (a) possess more than two labile hydrogen-containing functional groups, the resulting polyurethanes have a branched structure.

In a non-limiting embodiment of the at least one polyurethane according to the present disclosure, the tertiary and/or quaternary amines forming the cationic units (a) comprise only two labile hydrogen-containing reactive functional groups. Consequently, the polyurethanes resulting from the polycondensation have a substantially linear structure.

In another non-limiting embodiment, a mixture of difunctional amines comprising a small proportion of amines possessing more than two labile hydrogen-containing reactive functional groups may be used.

The tertiary or quaternary amines forming the cationic units (a) can be, for example, chosen from compounds corresponding to one of the following formulae:

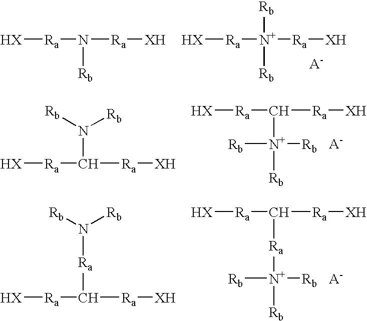

wherein

each Rₐ independently is chosen from linear and branched C₁₋₆ alkylene groups, C₃₋₆ cycloalkylene groups, and arylene groups, wherein all of which are optionally substituted with at least one halogen atom and/or comprise at least one heteroatom chosen from O, N, P, and S,

each R_{b} independently is chosen from C₁₋₆ alkyl groups, C₃₋₆cycloalkyl groups, and aryl groups, wherein all of which are optionally substituted with at least one halogen atom and/or comprise at least one heteroatom chosen from O, N, P, and S,

each X independently is chosen from an oxygen atom, a sulfur atom, an NH group, and NR_{c} groups, wherein R_{c} is a C₁₋₆ alkyl group, and

A⁻ represents a physiologically acceptable counter-ion.

Non-limiting examples of tertiary amines for producing the cationic polyurethanes include N-methyldiethanol amine and N-tert-butyldiethanol amine.

In at least one embodiment, tertiary and quaternary amines forming the cationic units (a) of the polyurethanes of the present disclosure may also be tertiary and/or quaternary amine function-containing polymers, possessing labile hydrogen-containing reactive functional groups at their ends. Weight average molecular weight of such tertiary and/or quaternary amine function-containing polymers ranges from 400 to 10,000.

Non-limiting examples of such amine function-containing polymers include polyesters resulting from the polycondensation of N-methyldiethanol amine and adipic acid.

When the amines forming the cationic units (a) are tertiary amine function compounds, all or part of these amine functional groups may be neutralized with a suitable neutralizing agent chosen from physiologically acceptable organic and mineral acids. Non-limiting examples of acids include hydrochloric acid and acetic acid.

In another embodiment, the second type of units forming the polyurethanes of the present disclosure include macromolecular units, called (b) units, derived from non-ionic polymers possessing labile hydrogen-containing reactive functional groups at their ends and having a glass transition temperature (Tg) less than 10° C., as measured by differential enthalpy analysis.

According to the present disclosure, at least one of these units (b1) is derived from olefinic homopolymers and/or copolymers possessing labile hydrogen-containing reactive functional groups at their ends.

For example, in at least one embodiment, polyurethane viscoelastic properties are achieved when (b) units are derived from polymers having a glass transition temperature less than 0° C., such as less than −10° C.

In at least one embodiment, the polyurethanes have a weight average molecular weight ranging from 400 to 10,000, such as from 1,000 to 5,000.

Non-ionic polymers that can form non-ionic units (b2), which are different from non-ionic units (b1), derive from olefinic homopolymers and/or copolymers, may be chosen from polyethers, polyesters, polysiloxanes, polycarbonates, and fluorinated polymers.

In at least one embodiment, polymers that can form non-ionic units (b) are only chosen from olefinic homopolymers and copolymers.

Non-limiting examples of olefinic polymers having labile hydrogen-containing reactive groups on their terminal ends, to be used in the present disclosure, include ethylene, propylene, 1-butylene, 2-butylene, isobutylene, 1,2-butadiene, 1,4-butadiene, and isoprene random or block homopolymers and copolymers.

Butadiene and isoprene homopolymers and copolymers may be partially or fully hydrogenated.

In at least one embodiment, the polymers are copolymers of ethylene/butylene, polybutadienes, and hydrogenated polybutadienes possessing on their terminal ends labile hydrogen-containing reactive groups, such as hydroxyl groups. In another embodiment, these polymers are 1,2- and/or 1,4-polybutadienes.

Such polymers are commercially available, for example, under the trade name KRATON® L, such as KRATON® L 2203 (hydrogenated polybutadiene diol) from the KRATON polymers company, KRASOL LBH® and LBHP®, such as KRASOL LBHP® 2000 (polybutadiene diol) from the SARTOMER company, and GI® 3000 (copolymer of ethylene and butylene) from the NISSO CHEMICAL company.

The diisocyanates forming the (c) units include aliphatic, alicyclic, or aromatic diisocyanates.

Non-limiting examples of diisocyanates include methylenediphenyl diisocyanate, methylenecyclohexane diisocyanate, isophorone diisocyanate, toluene diisocyanate, naphthalene diisocyanate, butane diisocyanate, and hexyl diisocyanate. These diisocyanates may be used alone or as a mixture of two or more diisocyanates. In at least one embodiment, the diisocyanate is isophorone diisocyanate.

As previously mentioned, cationic polyurethanes of the present disclosure may contain, in addition to (a), (b1), and (c) units, a certain amount of non-ionic units (b2) derived from compounds, that are generally monomer, non-ionic compounds, comprising at least two labile hydrogen functional groups, different from the compounds leading to the non-ionic units (b1).

These non-ionic units (b2) are generally derived from C₁-C₁₂ diols, for example from neopentyl glycol, hexaethylene glycol, 1,2-ethanediol, 1,2-propanediol, and 1,3-propanediol, or from C₁-C₆ aminoalcohols, for example from aminoethanol.

The cationic polyurethanes of the present disclosure can be, for example, elastic.

In at least one embodiment of the present disclosure, the at least one cationic polyurethane does not comprise any further unit in addition to the (a), (b), and (c) units. The polyurethane (A) described in the example section is a polyurethane encompassed within such a definition.

In an alternative embodiment, the at least one cationic polyurethane comprises further units in addition to the (a), (b), and (c) units. The polyurethane (B) described in the example section is a polyurethane encompassed within such a definition.

At least one physical parameter characterizing the viscoelastic properties of the at least one cationic polyurethane of the present disclosure is the tensile recovery. Such recovery is determined by a tensile creep test comprising rapidly stretching a specimen to a predetermined degree of elongation, then releasing the stress, and lastly measuring the specimen length.

The creep test used to characterize the cationic polyurethanes with elastic character of the present disclosure is performed as follows:

The specimen used is a film of polyurethane 500±50 mm-thick, cut into 80 mm×15 mm strips. This copolymer film is obtained by drying at a temperature of 22±2° C. under a 50±5% relative humidity, a 3% by weight solution or dispersion of said polyurethane in water and/or in ethanol.

Each strip is fixed between two jaws, spaced apart from each other by 50±1 mm, and is stretched at a speed of 20 mm/minute (under the hereabove mentioned temperature and relative humidity conditions) up to a 50% elongation (εₘₐₓ), that is to say until a strip is obtained, which size corresponds to 1.5 times its initial length. The stress is then released by setting a return speed equal to the tensile speed, i.e. 20 mm/minute, and the specimen elongation is then measured (as expressed in % relative to the initial length) immediately once it has returned to a zero load (εᵢ).

The instantaneous recovery (Rᵢ) is calculated using the following equation:

Rᵢ(%)=((εₘₐₓ−εᵢ)/εₘₐₓ)×100

The elastic cationic polyurethanes of the present disclosure can have, for example, an instantaneous recovery (Rᵢ), such as measured in the hereabove stated conditions ranging from 5% to 95%, such as from 20% to 90% and from 35 to 85%.

The glass transition temperature (Tg) of the non-ionic polymers forming the (b) units and of the cationic polyurethanes of the present disclosure is measured via a differential enthalpy analysis (DSC, differential scanning calorimetry) according to ASTM D3418-97 standard.

Elastic cationic polyurethanes of the present disclosure present at least two glass transition temperatures, at least one of which is less than 10° C., such as less than 0° C. and less than −10° C., the other one being at least greater than or equal to the room temperature (about 20° C.).

The instantaneous recovery and consequently the viscoelastic properties of the polyurethanes of the present disclosure depend on the contents of the various (a), (b1), (b2), and (c) monomer units.

The cationic unit (a) content should be sufficient to provide the polymers with their positive charge responsible for their good affinity for keratinic substrates. The non-ionic units (b) should be present in a weight content sufficient for the polyurethanes to have at least one glass transition temperature less than 10° C., and not to form brittle films.

In at least one embodiment the cationic units (a) are present in an amount ranging from 0.1% to 90%, such as from 1% to 30%, from 5% to 25%, and from 5% to 10% by weight; the non-ionic units (b1) are present in an amount ranging from 10% to 99.9%, such as from 20% to 99% and from 30% to 85% by weight; and the non-ionic units (b2) are present in an amount ranging from 0% to 50% by weight, such as from 0% to 30% by weight, of the total weight of the polyurethane units. In at least one embodiment, the polyurethanes of the present disclosure do not comprise any non-ionic unit (b2).

In at least one embodiment, the (c) units are present in an amount ranging from 1% to 60%, such as from 5% to 50%, and from 10% to 40% by weight of the total weight of the polyurethane units.

The (c) units may be present in a substantially stoichiometric amount as compared to the sum of (a) and (b) units. Obtaining polyurethanes with high molecular weights requires a number of isocyanate functional groups almost identical to the number of labile hydrogen functional groups. A person skilled in the art will be able to choose an optional molar excess of the one functional group or the other, to adjust the molecular weight to the desired value.

The amount of the at least one cationic polyurethane present in a cosmetic composition of the present disclosure depends on the composition type and the required properties and may vary within a very broad range, generally ranging from 0.01% to 40% by weight, such as from 0.05% to 20%, and from 0.1% to 10% by weight of the final cosmetic composition.

In at least one embodiment, the at least one polyethylene glycol ester is chosen from those of formula (I):

X—(OCH₂CH₂)ₘOY (I)

wherein

m ranges from 80 to 500, and

X and Y, independently from each other, are chosen from a hydrogen atom, and linear and branched, saturated and unsaturated C₈ to C₃₀ acyl groups,

with the proviso that at least one of the X and Y groups is an acyl group.

For example, polyethylene glycol comprising from 80 to 500 ethylene oxide groups per molecule may be used to produce the polyethylene glycol ester having the hereinabove formula (I).

Non-limiting polyethylene glycol examples include:

PEG-90 polyethylene glycol, comprising an average number of ethylene glycol groups of 90 and having a theoretical average molecular weight mW of about 4,000 g/mol,

PEG-100 polyethylene glycol, comprising an average number of ethylene glycol groups of 100 and having a theoretical average molecular weight mW of about 4,400 g/mol,

PEG-135 polyethylene glycol, comprising an average number of ethylene glycol groups of 135 and having a theoretical average molecular weight mW of about 6,000 g/mol,

PEG-150 polyethylene glycol, comprising an average number of ethylene glycol groups of 150 and having a theoretical average molecular weight mW of about 6,600 g/mol,

PEG-180 polyethylene glycol, comprising an average number of ethylene glycol groups of 180 and having a theoretical average molecular weight mW of about 7,900 g/mol,

PEG-200 polyethylene glycol, comprising an average number of ethylene glycol groups of 200 and having a theoretical average molecular weight mW of about 8,800 g/mol,

PEG-240 polyethylene glycol, comprising an average number of ethylene glycol groups of 240 and having a theoretical average molecular weight mW of about 10,600 g/mol,

PEG-350 polyethylene glycol, comprising an average number of ethylene glycol groups of 350 and having a theoretical average molecular weight mW of about 15,400 g/mol,

PEG-454 polyethylene glycol, comprising an average number of ethylene glycol groups of 454 and having a theoretical average molecular weight mW of about 20,000 g/mol.

The theoretical average molecular weight mW of a polyethylene glycol is calculated based on the following equation (I):

Theoretical mW=18+44×m (I)

where m ranges from 80 to 500.

In at least one embodiment, m ranges from 80 to 350, such as from 100 to 300.

In at least one embodiment, the ratio (R), defined as follows:

R=[hydrophilic part weight (OCH₂CH₂)ₘ]/[polyethylene glycol ester weight−hydrophilic part weight (OCH₂CH₂)ₘ],

ranges from 8 to 1,000.

Non-limiting examples of polyethylene glycol ester of formula (I) include polyethylene glycol monoesters, such as those products referred to in “International Cosmetic Ingredient Dictionary and Handbook” 9th Edition, 2002, under the references PEG90 STEARATE, PEG100 STEARATE, PEG120 STEARATE, PEG150 STEARATE, PEG150 LAURATE, and PEG150 OLEATE.

Non-limiting examples of polyethylene glycol diesters include products referred to in “International Cosmetic Ingredient Dictionary and Handbook” 9th Edition, 2002, under the references PEG150 DILAURATE, PEG150 DIOLEATE, PEG120 DISTEARATE, PEG150 DISTEARATE, PEG175 DISTEARATE, and PEG250 DISTEARATE.

The at least one polyethylene glycol ester may be a monoester or a diester. In at least one embodiment, the polyethylene glycol ester is a diester.

The amount of the at least one polyethylene glycol ester in a cosmetic composition according to the present disclosure depends on the composition type and on the desired properties and may vary within a very broad range, typically from 0.01% to 50% by weight, such as from 0.1% to 20%, and from 0.5% to 15% by weight of the weight of the final cosmetic composition. It is understood that at least one polyethylene glycol ester type is incorporated into the cosmetic composition. For example, a monoester and a diester may be incorporated into the cosmetic composition of the present disclosure.

In at least one embodiment, the cosmetically acceptable medium is an aqueous medium.

The cosmetic composition of the present disclosure may also comprise at least one organic solvent, for example, in an amount ranging from 0.05% to 40%, such as from 1% to 20% by weight of the composition total weight.

The at least one organic solvent may be a lower C₂-C₄ alcohol, such as ethanol, a polyol, such as propylene glycol or glycerol, or a polyol ether.

The compositions of the present disclosure may also comprise at least one cosmetically acceptable additive/adjuvant, such as for example surfactants (which are different from a polyethylene glycol ester as some polyethylene glycol esters may be surfactants), thickeners, penetrating agents, fragrances, buffering agents, and various usual additives such as UV absorbing filters, waxes, cyclic, linear or branched, volatile or non volatile silicones, either organomodified (such as modified with amine groups) or not, preserving agents, ceramids, pseudoceramids, mineral, vegetable or synthetic oils, vitamins or provitamins, such as panthenol, opacifying agents, reducing agents, emulsifying agents, preservatives, fillers, sunscreen agents, proteins, anionic, non-ionic, cationic or amphoteric fixing polymers, moisturizing agents, emollients, softening agents, anti-foam agents, antiperspirants, anti-free radical agents, fixing or non fixing polymers, bactericides, sequestering agents, anti-dandruff agents, antioxidants, alkalinizing agents, and any other additive/adjuvant traditionally used in cosmetic compositions intended to be applied onto the hair.

The surfactants, which are different from the polyethylene glycol esters that may be used in the composition of the present disclosure, may be anionic, non-ionic, amphoteric, or cationic surfactants, or mixtures thereof.

Suitable anionic surfactants to be used either alone or in combination in the context of the present disclosure include, for example, salts: alkaline metal salts such as sodium salts, ammonium salts, amine salts, aminoalcohol salts or alkaline-earth metal salts, for example, magnesium salts, of following compounds: alkyl sulfates, alkyl ethersulfates, alkyl amidoethersulfates, alkylaryl polyethersulfates, monoglyceride sulfates; alkyl sulfonates, alkyl amidesulfonates, alkyl-aryl sulfonates, α-olefin sulfonates, paraffin sulfonates; alkyl sulfosuccinates, alkyl ethersulfosuccinates, alkylamide sulfosuccinates; alkyl sulfoacetates; acyl sarconisates; and acylglutamates, the alkyl and acyl groups of all these compounds comprise from 6 to 24 carbon atoms, and the aryl group corresponds to a phenyl or benzyl group.

Polyglycoside carboxylic acid and C₆-C₂₄ alkyl esters may also be used in the context of the present disclosure, such as alkyl glucoside citrates, alkyl polyglycoside tartrates, and alkyl polyglycoside sulfosuccinates; as well as alkyl sulfosuccinamates, acyl isethionates, and N-acyl taurates, the alkyl or acyl group of all these compounds comprises from 12 to 20 carbon atoms. Additional anionic surfactants include, acyl lactylates, the acyl group of which comprises from 8 to 20 carbon atoms.

Alkyl-D-galactoside uronic acids and salts thereof may also be used in the compositions of the present disclosure, as well as polyoxyalkylene (C₆-C₂₄)alkylether carboxylic acids, polyoxyalkylene (C₆-C₂₄)alkyl(C₆-C₂₄)arylether carboxylic acids, polyoxyalkylene (C₆-C₂₄)alkylamidoether carboxylic acids, and salts thereof, such as those comprising from 2 to 50 ethylene oxide groups, and mixtures thereof.

Among the above mentioned anionic surfactants, further non-limiting mention may be made of (C₆-C₂₄)alkyl sulfates, (C₆-C₂₄)alkyl ethersulfates, (C₆-C₂₄)alkyl ethercarboxylates, and mixtures thereof, for example ammonium lauryl sulfate, sodium lauryl sulfate, magnesium lauryl sulfate, sodium lauryl ethersulfate, ammonium lauryl ethersulfate, and magnesium lauryl ethersulfate.

The composition of the present disclosure may comprise at least one anionic surfactant in an amount ranging from 0.5% to 60% by weight, such as from 5% to 20% by weight of the composition total weight.

Non-ionic surfactants to be used in the context of the present disclosure include compounds that are well known (for a review thereof, see, for instance, “Handbook of Surfactants” M. R. PORTER, Blackie & Son Editor (Glasgow and London), 1991, pp 116-178). The at least one non-ionic surfactant may be chosen from alcohols, alpha-diols, (C₁-C₂₀)alkyl phenols, and polyethoxylated, polypropoxylated, and polyglycerolated fatty acids, having a fatty-chain comprising for example from 8 to 18 carbon atoms, where the number of ethylene oxide or propylene oxide groups may range from 2 to 50, and the number of glycerol groups may range from 2 to 30. Other non-limiting examples include copolymers of ethylene oxide and propylene oxide, condensation products of ethylene oxide and propylene oxide on fatty alcohols; polyethoxylated fatty amides having, for example, from 2 to 30 moles of ethylene oxide; polyglycerolated fatty amides comprising on average from 1 to 5 glycerol groups, such as from 1.5 to 4 glycerol groups; polyethoxylated fatty amines having, for example, from 2 to 30 moles of ethylene oxide; sorbitane fatty acid esters ethoxylated with from 2 to 30 moles of ethylene oxide; sucrose fatty acid esters, polyethylene glycol fatty acid esters, (C₆-C₂₄)alkyl polyglucosides, (C₆-C₂₄)N-alkyl glucamine derivatives, amine oxides such as (C₁₀-C₁₄)alkyl amine oxides and (C₁₀-C₁₄)N-acyl aminopropylmorpholine oxides.

In at least one embodiment, the at least one non-ionic surfactant is chosen from (C₆-C₂₄)alkyl polyglycosides, such as decyl polyglucoside.

Among the amphoteric surfactants to be used in the present disclosure, non-limiting mention may be made of secondary and tertiary aliphatic amine derivatives, wherein the aliphatic group is a linear or a branched chain comprising from 8 to 22 carbon atoms and containing at least one hydrosolubilizing anionic group, such as a carboxylate, sulfonate, sulfate, phosphate, or phosphonate group; (C₈-C₂₀)alkyl betaines, sulfobetaines, (C₈-C₂₀)alkyl(C₆-C₈)amidoalkyl betaines, or (C₈-C₂₀)alkyl(C₆-C₈)amidoalkyl sulfobetaines; as well as mixtures thereof.

Non-limiting examples of amine derivatives include products marketed under the trade name MIRANOL®, such as those described in U.S. Pat. Nos. 2,528,378 and 2,781,354, and classified in the CTFA dictionary, third Edition, 1982, under the names amphocarboxyglycinate and amphocarboxypropionate having respectively the following structures (1) and (2):

R₂—CONHCH₂CH₂—N⁺(R₃)(R₄)(CH₂COO⁻) (1)

wherein:

R₂ is an alkyl group derived from a R₂—COOH acid present in hydrolyzed coconut oil, a heptyl, nonyl or undecyl group,

R₃ is a beta-hydroxyethyl group, and

R₄ is a carboxymethyl group; and

R₂—CONHCH₂CH₂—N(B)(C) (2)

wherein:

B is —CH₂CH₂OX′,

C is —(CH₂)_{z}—Y′, with z=1 or 2,

X′ is a —CH₂CH₂—COOH group or a hydrogen atom,

Y′ is —COOH or a —CH₂—CHOH—SO₃H group,

R₂ is the alkyl group of a R₂—COOH acid present in hydrolyzed coconut oil or linseed oil, an alkyl group, such as a C₁₇ alkyl group and its iso-form, an unsaturated C₁₇ group.

These compounds are classified in the CTFA dictionary, 5th Edition, 1993, under the names disodium cocoamphodiacetate, disodium lauroamphodiacetate, disodium capryl amphodiacetate capryloamphodiacetate, disodium cocoamphodipropionate, disodium lauroamphodipropionate, disodium caprylamphodipropionate, disodium capryloamphodipropionate, lauroamphodipropionic acid, and cocoamphodipropionic acid.

The cocoamphodiacetate marketed under the trade name MIRANOL® C2M concentrated by the RHODIA company is a suitable non-limiting example thereof.

Non-limiting examples of amphoteric surfactants include (C₈-C₂₀)alkyl betaines, such as coco betaine, (C₈-C₂₀)alkyl(C₆-C₈)amidoalkyl betaines, such as cocamido betaine, alkyl amphodiacetates, such as disodium cocoamphodiacetate, and mixtures thereof.

The composition of the present disclosure may further comprise at least one cationic surfactant, such as salts of primary, secondary, or tertiary fatty amines, optionally polyoxyalkylenated, quaternary ammonium salts, such as tetraalkylammonium, alkylamidoalkyl trialkylammonium, trialkylbenzylammonium, trialkylhydroxyalkylammonium, or alkylpyridinium chlorides, or bromides, imidazoline derivatives; or amine oxides of cationic nature.

The previously described non-ionic, amphoteric, and cationic surfactants may be used either alone or in combination, and they may be present in an amount ranging from 0.1% to 30% by weight, such as from 0.5% to 25% and from 1% to 20% by weight relative to the composition total weight.

Non-limiting examples of gelling agents and/or thickeners that may be used in the compositions of the present disclosure are known in the art and may be chosen from, for example, carboxyvinyl polymers and copolymers, (alkyl)acrylic polymers and copolymers, (alkyl)acrylamide polymers and copolymers, poly(oxyalkylene)glycols, poly(oxyalkylene)glycol esters, alginates, biosaccharides, polysaccharides, such as cellulose and starch derivatives, naturally occurring gums, such as xanthan gum, guar gum, locust bean gum, scleroglucans, chitin and chitosan derivatives, carrageenans, clays, and mixtures thereof.

Non-limiting examples of gelling agents, such as in an aqueous phase, include SEPIGEL® 305 marketed by the SEPPIC company, FUCOGEL® 1000 PP marketed by the SOLABIA company, SYNTHALEN® K marketed by the 3VSA company, LUVISKOL® VA 64 P marketed by the BASF company, HOSTACERIN® AMPS marketed by the CLARIANT company, PEMULEN® TR1 marketed by the GOODRICH company, LUBRAGEL® MS marketed by the GUARDIAN company, SATIAGEL® KSO marketed by DEGUSSA and KELTROL® marketed by the KELCO.

The at least one gelling agent may be present in an amount ranging from 0.05% to 15% by weight, such as from 0.5% to 10% by weight of the composition.

The at least one silicone that may be used as an additive in the cosmetic compositions of the present disclosure is chosen from volatile and non volatile, cyclic, linear and branched silicones, unmodified and modified with organic groups, and having a viscosity ranging from 5.10⁻⁶ m²/s to 2.5 m²/s at 25° C., such as from 1.10⁻⁵ m²/s to 1 m²/s.

The at least one silicone that can be used according to the present disclosure may be soluble or insoluble in the composition, such as polyorganosiloxanes that are insoluble in the composition of the present disclosure. They may be present in the form of oils, waxes, resins, or gums.

Organopolysiloxanes are defined in more detail by Walter NOLL in “Chemistry and Technology of Silicones” (1968) Academie Press. They may be volatile or non-volatile.

When the organopolysiloxanes are volatile, the silicones are chosen from those having a boiling point ranging from 60° C. to 260° C., including:

(i) cyclic silicones comprising from 3 to 7 silicon atoms, such as from 4 to 5 silicon atoms. Suitable examples thereof include octamethyl cyclotetrasiloxane marketed under the trade name “VOLATILE SILICONE® 7207” by UNION CARBIDE or “SILBIONE® 70045 V 2” by RHODIA, decamethyl cyclopentasiloxane marketed under the trade name “VOLATILE SILICONE® 7158” by UNION CARBIDE, “SILBIONE® 70045 V 5” by RHODIA, as well as mixtures thereof.

Other organopolysiloxanes include cyclocopolymers of the dimethyl siloxane and methylalkyl siloxane type, such as “SILICONE VOLATILE® FZ 3109” marketed by the UNION CARBIDE company, having following formula:

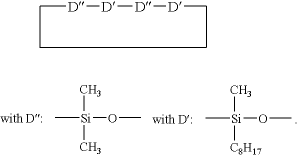

Cyclic silicones with organic compounds derived from silicon, such as the octamethyl cyclotetrasiloxane and tetratrimethylsilyl pentaerythritol mixture (50:50) and the octamethyl cyclotetrasiloxane, and oxy-1,1′-(hexa-2,2,2′,2′,3,3′-trimethylsilyloxy)bis-neopentane mixture; and

(ii) linear volatile silicones having from 2 to 9 silicon atoms wherein the viscosity is less than or equal to 5.10⁻⁶ m²/s at 25° C., as for example decamethyl tetrasiloxane marketed, for instance, under the trade name “SH 200” by the TORAY SILICONE company may also be used according to the present disclosure. Silicones belonging to this class are also described in the article published in Cosmetics and Toiletries, Vol. 91, Jan. 76, P. 27-32—TODD & BYERS “Volatile Silicone fluids for cosmetics”.

Non volatile silicones may be used, such as polyalkyl siloxanes, polyaryl siloxanes, polyalkylaryl siloxanes, silicone-based gums and resins, polyorganosiloxanes modified with organofunctional groups, as well as mixtures thereof.

These silicones may be chosen from polyalkyl siloxanes, including polydimethyl siloxanes with trimethylsilyl end groups. Silicone viscosity is measured at 25° C. according to ASTM 445 standard, Appendix C.

These polyalkyl siloxanes encompass, as non-limiting examples, the following commercial products:
SILBIONE® oils of 47 and 70 047 series or MIRASIL® oils marketed by RHODIA, such as for example the oil 70 047 V 500 000;
oils of MIRASIL® series marketed by the RHODIA company;
oils of the 200 series from the DOW CORNING company, such as DC200 (viscosity 60,000 mm²/s); and
VISCASIL® oils from GENERAL ELECTRIC and certain oils of the SF (SF 96, SF 18) series from GENERAL ELECTRIC.

Dimethylsilanol end group-containing polymethyl siloxanes, known under the name dimethiconol (CTFA) may also be used, such as the oils of the 48 series from the RHODIA company.

This polyalkyl siloxane class also includes products marketed under the trade names “ABIL WAX® 9800 and 9801” by the GOLDSCHMIDT company, which are (C₁-C₂₀) polyalkyl siloxanes.

Polyalkylaryl siloxanes may be chosen from linear and/or branched polydimethyl/methylphenyl siloxanes, polydimethyl/diphenyl siloxanes having viscosities ranging from 1.10⁻⁵ m²/s to 5.10⁻² m²/s at 25° C.

Suitable examples of such polyalkylaryl siloxanes include products marketed under the following trade names:
SILBIONE® oils of the 70 641 series from RHODIA;
oils of RHODORSIL® 70 633 and 763 series from RHODIA;
DOW CORNING 556 COSMETIC GRAD FLUID from DOW CORNING;
silicones of the PK series from BAYER, such as the PK20 product;
silicones of the PN, PH series from BAYER, such as PN1000 and PH1000 products; and
Certain oils of the SF series from GENERAL ELECTRIC, such as SF 1023, SF 1154, SF 1250, SF 1265.

Non-limiting examples of silicone gums to be used according to the present disclosure include polyorganosiloxanes having high number average molecular weights ranging from 200,000 to 1,000,000 and may be used either alone or in combination with a solvent. This solvent may be chosen from volatile silicones, polydimethyl siloxane (PDMS) oils, polyphenylmethyl siloxane (PPMS) oils, isoparaffins, polyisobutylenes, methylene chloride, pentane, dodecane, and tridecane.

Non-limiting examples of silicone gums include the following:

Polydimethyl siloxane,

Polydimethyl siloxane/methylvinyl siloxane gums,

Polydimethyl siloxane/diphenyl siloxane,

Polydimethyl siloxane/phenylmethyl siloxane, and

Polydimethyl siloxane/diphenyl siloxane/methylvinyl siloxane.

Other non-limiting products to be used according to the present disclosure include mixtures such as

mixtures formed from an end chain-hydroxylated polydimethyl siloxane also called dimethiconol (CTFA) and a cyclic polydimethyl siloxane, also called cyclomethicone (CTFA), such as the Q2 1401 product marketed by the DOW CORNING company;

mixtures formed from a polydimethyl siloxane gum and a cyclic silicone, such as the SF 1214 Silicone Fluid from the GENERAL ELECTRIC company, this product being a SF 30 gum corresponding to a dimethicone, having a number average molecular weight of 500,000, solubilized in the SF 1202 Silicone Fluid corresponding to decamethyl cyclopentasiloxane; and

mixtures from two PDMS with different viscosities, and for example, from a PDMS gum and a PDMS oil, such as the SF 1236 product from GENERAL ELECTRIC. SF 1236 is a mixture from a SE 30 gum as defined hereabove with a viscosity of 20 m²/s and a SF 96 oil with a viscosity of 5.10⁻⁶ m²/s. Such product comprises about 15% of SE 30 gum and 85% of SF 96 oil.

Other non-limiting examples of organopolysiloxane resins to be used according to the present disclosure include crosslinked siloxane systems comprising R₂SiO_{1/2}, R₃SiO_{1/2}, RSiO_{3/2} and SiO_{4/2} units, wherein R is a hydrocarbon group having from 1 to 16 carbon atoms or a phenyl group. In at least one embodiment, R is a lower C₁-C₄ alkyl group, such as a methyl group, or R is a phenyl group.

These resins also include the product marketed under the trade name “DOW CORNING 593” or those marketed under the trade names “SILICONE FLUID SS 4230 and SS 4267” by the GENERAL ELECTRIC company and which are dimethyl/trimethyl siloxane-structured silicones.

Resins of the trimethyl siloxysilicate type marketed for example under the trade names X22-4914, X21-5034 and X21-5037 by the SHIN-ETSU company may also be used according to the present disclosure.

Non-limiting examples of organomodified silicones to be suitably used according to the present disclosure are silicones such as previously defined, and comprising in their structure at least one organofunctional group bound through a hydrocarbon group.

Non-limiting examples of organomodified silicones to be suitably used according to the present disclosure include polyorganosiloxanes comprising:

polyethyleneoxy and/or polypropyleneoxy groups optionally comprising C₆-C₂₄ alkyl groups, such as products called dimethicone copolyol marketed by the DOW CORNING company under the trade name DC 1248 or SILWET® L 722, L 7500, L 77, L 711 oils from the UNION CARBIDE company and (C₁₋₂)alkyl methicone copolyol marketed by the DOW CORNING company under the trade name Q2 5200;

amine groups, substituted or unsubstituted, such as the products marketed under the trade name GP 4 Silicone Fluid and GP 7100 by the GENESEE company, or the products marketed under the trade names Q2 8220 and DOW CORNING 929 or 939 by the DOW CORNING company. Substituted amine groups include C₁-C₄ aminoalkyl groups;

thiol groups, such as the products marketed under the trade names GP 72A and GP 71 from GENESEE;

alkoxyl groups, such as the product marketed under the trade name “SILICONE COPOLYMER F-755” by SWS SILICONES and ABIL WAX® 2428, 2434 and 2440 by the GOLDSCHMIDT company;

hydroxyl groups, such as the hydroxyalkyl functional group-containing polyorganoxiloxanes described in the French patent application FR-A-8 516 334;

alkoxyalkyl groups, such as for example the polyorganosiloxanes described in patent U.S. Pat. No. 4,957,732;

carboxylic type anionic groups, such as for example, in products described in European patent EP 186 507 from the CHISSO CORPORATION company, or alkyl carboxylic type anionic groups such as those comprised in the X-22-3701 E product from the SHIN-ETSU company; or 2-hydroxyalkyl sulfonate; 2-hydroxyalkyl thiosulfate such as the products marketed by the GOLDSCHMIDT company under the trade names ABIL® S201 and ABIL® S255; and

hydroxyacrylamino groups, such as the polyorganosiloxanes described in the European patent application EP 342 834. The Q2-8413 product from the DOW CORNING company is an example thereof.

The silicones such as described hereabove may be used either alone or in combination, in an amount ranging from 0.01% to 20% by weight, such as from 0.1% to 5% by weight relative to the total weight of the composition.

The compositions of the present disclosure may also comprise fatty components such as mineral, vegetable, animal and synthetic oils, waxes, fatty esters, fatty alcohols, and fatty acids.

Non-limiting examples of oils to be used in the composition of the disclosure include the following:

animal-based hydrocarbon oils, such as perhydrosqualene;

vegetable-based hydrocarbon oils, such as liquid triglycerides of fatty acids comprising from 4 to 10 carbon atoms such as triglycerides of the heptanoic or octanoic acids, or for example sunflower oil, corn oil, soja bean oil, pumpkin oil, grape seed oil, sesame oil, nut oil, apricot kernel oil-, macadamia nut oil, arara oil, castor oil, avocado oil, triglycerides of caprylic/capric acids such as those marketed by the Stearineries Dubois company or those sold under the names Miglyol® 810, 812 and 818 by the Dynamit Nobel company, jojoba oil, shea butter oil;

linear or branched, mineral or synthetic hydrocarbons, such as volatile or non volatile paraffin oils, and their derivatives, petrolatum, polydecenes, hydrogenated polyisobutene such as Parleam®; isoparaffines such as isohexadecane and isodecane; and

partly hydrocarbon-based and/or silicone-based fluorinated oils, such as those described in the patent application JP-A-2-295912; fluorinated oils also encompass perfluoromethyl cyclopentane and perfluoro-1,3 dimethylcyclohexane, sold under the names “FLUTEC® PC1” and “FLUTEC® PC3” by the BNFL Fluorochemicals company; perfluoro-1,2-dimethyl cyclobutane; perfluoroalkanes such as dodecafluoropentane and tetradecafluorohexane, sold under the names “PF 5050®” and “PF 5060®” by the 3M company, or bromoperfluorooctyl sold under the trade name “FORALKYL®” by the Atochem company; nonafluoromethoxybutane and nonafluoroethoxyisobutane; perfluoromorpholine derivatives, such as 4-trifluoromethyl perfluoromorpholine sold under the trade name “PF 5052®” by the 3M company.

Non-limiting examples of waxes include Carnauba wax, Candellila wax, and Alfa wax, paraffin, ozokerite, vegetable waxes, such as olive tree wax, rice wax, hydrogenated jojoba wax or flower absolute waxes, such as Ribes nigrum (blackcurrant) flower wax sold by the BERTIN company (France), animal waxes, such as beeswax, or modified beeswaxes (cerabellina); other waxes or wax-based raw materials to be used according to the present disclosure also include marine waxes, such as the one sold by the SOPHIM company under the reference M82, polyethylene waxes, or polyolefins.

Saturated or unsaturated fatty acids may be chosen from myristic acid, palmitic acid, stearic acid, behenic acid, oleic acid, linoleic acid, linolenic acid, and isostearic acid.

Non-limiting fatty esters include carboxylic acid esters, such as mono-, di-, tri- or tetracarboxylic esters.

Carboxylic acid esters may be saturated or unsaturated, linear or branched, C₁-C₂₆ aliphatic acid esters, and saturated or unsaturated, linear or branched, C₁-C₂₆ aliphatic alcohol esters, wherein the total number of the ester carbon atoms is greater than or equal to 10.

Non-limiting examples of monoesters include dihydroabietyl behenate, octyidodecyl behenate, isocetyl behenate, cetyl lactate; C₁₂-C₁₅ alkyl lactate, isostearyl lactate, lauryl lactate, linoleyl lactate, oleyl lactate, (iso)stearyl octanoate, isocetyl octanoate, octyl octanoate, cetyl octanoate, decyl oleate, isocetyl isostearate, isocetyl laurate, isocetyl stearate, isodecyl octanoate, isodecyl oleate, isononyl isononanoate, isostearyl palmitate, methylacetyl ricinoleate, myristyl stearate, octyl isononanoate, 2-ethylhexyl isononate, octyl palmitate, octyl pelargonate, octyl stearate, octyldodecyl erucate, oleyl erucate, ethyl and isopropyl palmitates, ethyl-2-hexyl palmitate, 2-octyidecyl palmitate, alkyl myristates, such as isopropyl-, butyl-, cetyl-, and 2-octyldodecyl myristate, hexyl stearate, butyl stearate, isobutyl stearate, dioctyl malate, hexyl laurate, and 2-hexyldecyl laurate.

C₄-C₂₂ di- or tricarboxylic acid and C₁-C₂₂ alcohol esters may also be used, as well as mono-, di-, or tricarboxylic acid esters and di-, tri-, tetra-, or pentahydroxy C₂-C₂₆ alcohol esters.

Other non-limiting examples of monoesters include diethyl sebacate, diisopropyl sebacate, diisopropyl adipate, di n-propyl adipate, dioctyl adipate, diisostearyl adipate, dioctyl maleate, glyceryl undecylenate, octyldodecyl stearoyl stearate, pentaerythrityl monoricinoleate, pentaerythrityl tetraisononanoate, pentaerythrityl tetrapelargonate, pentaerythrityl tetraisostearate, pentaerythrityl tetraoctanoate, propylene glycol dicaprylate, propylene glycol dicaprate, tridecyl erucate, triisopropyl citrate, triisostearyl citrate, glyceryl trilactate, glyceryl trioctanoate, trioctyldodecyl citrate, trioleyl citrate, propylene glycol dioctanoate, and neopentyl glycol diheptanoate. The hereinabove mentioned esters are different from the esters of formula (I).

Among the esters mentioned above, at least one embodiment of the present disclosure includes ethyl- and isopropyl palmitates, ethyl-2-hexyl palmitate, 2-octyidecyl palmitate, alkyl myristates, such as isopropyl-, butyl-, cetyl-, and 2-octyldodecyl myristate, hexyl stearate, butyl stearate, isobutyl stearate, dioctyl malate, hexyl laurate, 2-hexyldecyl laurate, isononyl isononanate, and cetyl octanoate.

Non-limiting examples of fatty alcohols include saturated or unsaturated, linear or branched fatty alcohols having from 8 to 26 carbon atoms, such as cetyl alcohol, stearyl alcohol and a mixture thereof (cetylstearyl alcohol), octyldodecanol, 2-butyloctanol, 2-hexyldecanol, 2-undecylpentadecanol, oleic alcohol, and linoleic alcohol.

Fatty components are generally present in an amount ranging from 0.1% to 50%, such as from 1% to 30% and from 2% to 20% by weight of the total composition.

The person skilled in the art will recognize those additives/adjuvants and amounts thereof that are appropriate without affecting the desired and beneficial properties of the compositions of the present disclosure.

The compositions of the present disclosure may be present in any suitable form to be used for topical application, such as solutions of lotion or serum type; as aqueous gels; as emulsions obtained by dispersing a fatty phase into an aqueous phase (oil-in-water emulsion) or vice versa (water-in-oil emulsion), having a thick liquid consistancy, such as creamy cosmetic milks and creams; as foams, as sprays or aerosols; or as a brush or a stick.

When the composition of the present disclosure is conditioned in an aerosol-type device, it further comprises at least one propellant that may be chosen from volatile hydrocarbons, such as n-butane, propane, isobutane, pentane, halogenated hydrocarbons and mixtures thereof. Other non-limiting examples of propellants include carbon dioxide, nitrous oxide, dimethyl ether (DME), nitrogen, and compressed air. Mixtures of propellants may also be used. In at least one embodiment, dimethyl ether will be used.

The at least one propellant may be present in an amount ranging from 5% to 90% by weight of the composition total weight in the aerosol device, such as from 10% to 60% by weight of the composition total weight.

The composition of the present disclosure may be applied to the hair and not rinsed off.

Another aspect of the present disclosure is a method for treating keratinic materials comprising applying a cosmetic composition of the present disclosure. Still another aspect of the present disclosure is a hair reshaping method, comprising applying a cosmetic composition of the present disclosure.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

By way of non-limiting illustration, concrete examples of certain embodiments of the present disclosure are given below.

### EXAMPLES

### Example 1

### Styling Gel of the Present Disclosure

The composition was prepared according to the formulation in Table 1 (the amounts are expressed by weight, relative to the composition total weight):

TABLE 1

| | |
|---|---|
| Cationic polyurethane containing a | 6% |
| polyolefin sequence⁽¹⁾ | |
| Polyethylene glycol 6000 | 3% |
| distearate⁽²⁾ | |
| Simulsol 220 TM⁽³⁾ | 2% |
| Propylene glycol | 2.5% |
| Jaguar HP 105⁽⁴⁾ | 1% |
| Demineralized water | QS 100% |

⁽¹⁾(A) Polyurethane in an aqueous dispersion formed from 8.7% of N-methyl diethanol amine, 23.4% of isophorone diisocyanate, 67.9% of KRASOL LBH2000 (polybutadiene with hydroxyl end functions), and neutralized up to 40% using hydrogen chloride.
⁽²⁾Polyethylene glycol distearate marketed by the AKZO NOBEL company (PEG150 DISTEARATE)
⁽³⁾Oxyethylene glyceryl monostearate marketed by the SEPPIC company.
⁽⁴⁾Hydroxypropyl guar, marketed by the RHODIA company.

When applied to the hair, this gel made it possible to style the hair with a long-lasting hold, while resisting deterioration due to moisture, water, and to mechanical stresses. The gel tacky feel did not remain on the hands and was easily removed from the hair with shampoo.

With the gel formulation of Table 1, a long-lasting hair plastering down was obtained on African hair. After removal of the gel with shampoo a pleasant, smooth, cosmetic feel resulted. A good resistance to moisture, water, and to mechanical stresses was also observed.

A similar result was obtained with a (B) polyurethane in an aqueous dispersion formed from 8.4% of poly(tetramethylene oxide), 8.6% of N-methyl diethanol amine, 21.4% of isophorone diisocyanate, 61.6% of KRATON L2203 (polybutadiene with hydroxyl end functions), and neutralized up to 40% using hydrogen chloride.

What is claimed is:

1. A cosmetic composition comprising, in a cosmetically acceptable aqueous medium:
(i) at least one cationic polyurethane comprising at least one non-ionic unit derived from at least one polymer chosen from olefinic homopolymers and copolymers, and
(ii) at least one polyethylene glycol ester.

2. A cosmetic composition according to claim 1, wherein at least 50% by weight of the polyurethane non-ionic units are derived from at least one polymer chosen from olefinic homopolymers and copolymers.

3. A cosmetic composition according to claim 2, wherein all the polyurethane non-ionic units are derived from at least one polymer chosen from olefinic homopolymers and copolymers.

4. A cosmetic composition according to claim 1, wherein the olefinic homopolymers and copolymers possess labile hydrogen functional groups at their ends, and comprise units chosen from ethylene, propylene, 1-butylene, 2-butylene, isobutylene, 1,2-butadiene, 1,4-butadiene, and isoprene units.

5. A cosmetic composition according to claim 4, wherein the olefinic homopolymers and copolymers are derived from optionally hydrogenated 1,2-butadiene, and/or 1,4-butadiene.

6. A cosmetic composition according to claim 1, wherein the at least one cationic polyurethane comprises:
(a) cationic units (a) resulting from the reaction of at least one amine chosen from tertiary and quaternary amines comprising at least two labile hydrogen-containing reactive functional groups,
(b) non-ionic units, wherein at least one unit (b1) results from the reaction of at least one polymer chosen from olefinic homopolymers and copolymers possessing labile hydrogen-containing reactive functional groups at their ends and having a glass transition temperature (Tg) less than 10° C., and
(c) units (c) derived from at least one diisocyanate.

7. A cosmetic composition according to claim 6, wherein the cationic units (a) result from the reaction of at least one amine chosen from tertiary and quaternary amines comprising two labile hydrogen-containing reactive functional groups.

8. A cosmetic composition according to claim 7, wherein the at least one amine is chosen from amines of the formulae: 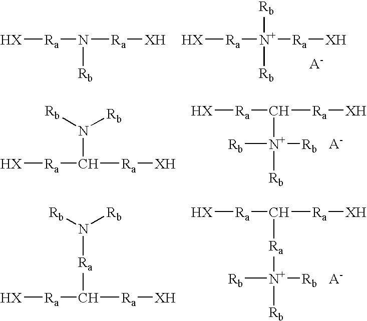
wherein
each Rₐ independently is chosen from linear and branched C₁-C₆ alkylene groups, C₃-C₆ cycloalkylene groups, and arylene groups; wherein all of which are optionally substituted with at least one halogen atom and/or comprise at least one heteroatom chosen from O, N, P, and S;
each R_{b} independently is chosen from C₁-C₆ alkyl groups, C₃-C₆ cycloalkyl groups, and aryl groups; wherein all of which are optionally substituted with at least one halogen atom and/or comprise at least one heteroatom chosen from O, N, P, and S;
each X independently is chosen from an oxygen atom, a sulfur atom, an NH group, and NR_{c} groups, wherein R_{c} is a C₁-C₆ alkyl group; and
A⁻ is a physiologically acceptable counter-ion.

9. A cosmetic composition according to claim 8, wherein the cationic units (a) result from the reaction of N-methyldiethanol amine and/or N-tert-butyldiethanol amine.

10. A cosmetic composition according to claim 7, wherein the cationic units (a) result from the reaction of at least one polymer chosen from tertiary and/or quaternary amine function-containing polymers, possessing labile hydrogen-containing reactive functional groups at their ends chosen from —OH, —NH₂, —NHR_{c}, and —SH, and having a weight average molecular weight ranging from 400 to 10,000, wherein R_{c} is a C₁-C₆ alkyl group.

11. A cosmetic composition according to claim 6, wherein the at least one cationic polyurethane optionally comprises at least one non-ionic unit (b2), which is different from the non-ionic unit (b1), wherein (b2) is derived from a non-ionic monomer compound comprising at least two labile-hydrogen functional groups that can react with the (c) compounds derived from at least one diisocyanate.

12. A cosmetic composition according to claim 11, wherein the cationic units (a) are present in an amount ranging from 0.1% to 90% by weight; the non-ionic units (b1) are present in an amount ranging from 10% to 99.9% by weight; and the non-ionic units (b2) are present in an amount ranging from 0% to 50% by weight of the cationic polyurethane total units.

13. A cosmetic composition according to claim 11, wherein the cationic units (a) are present in an amount ranging from 1% to 20% by weight; the non-ionic units (b1) are present in an amount ranging from 30% to 85% by weight of the cationic polyurethane total units; and the non-ionic units (b2) are present in an amount ranging from 0% to 30% by weight of the cationic polyurethane total units.

14. A cosmetic composition according to claim 6, wherein the at least one diisocyanate is chosen from methylenediphenyl diisocyanate, methylenecyclohexane diisocyanate, isophorone diisocyanate, toluene diisocyanate, naphthalene diisocyanate, 1,4-butane diisocyanate, and 1,6-hexane diisocyanate.

15. A cosmetic composition according to claim 14, wherein the at least one diisocyanate is isophorone diisocyanate.

16. A cosmetic composition according to claim 6, wherein the units (c) are present in an amount ranging from 1% to 60% by weight of the cationic polyurethane total units.

17. A cosmetic composition according to claim 16, wherein the units (c) are present in an amount ranging from 1% to 40% by weight of the cationic polyurethane total units.

18. A cosmetic composition according to claim 11, wherein the at least one non-ionic monomer compound forming the non-ionic units (b2) are chosen from C₁-C₁₂ diols and C₁-C₆ aminoalcohols.

19. A cosmetic composition according to claim 18, wherein the C₁-C₁₂ diol is chosen from neopentyl glycol, hexa(ethlyene glycol), 1,2-ethanediol, 1,2-propanediol, and 1,3-propanediol.

20. A cosmetic composition according to claim 18, wherein the C₁-C₆ aminoalcohol is aminoethanol.

21. A cosmetic composition according to claim 6, wherein the cationic polyurethane does not comprise any further unit in addition to the (a), (b), and (c) units.

22. A cosmetic composition according to claim 6, wherein the at least one cationic polyurethane is of the elastic type.

23. A composition according to claim 1, wherein the at least one cationic polyurethane is present in an amount ranging from 0.01% to 40% by weight relative to the final cosmetic composition.

24. A composition according to claim 23, wherein the at least one cationic polyurethane is present in an amount ranging from 0.1% to 10% by weight of the final cosmetic composition.

25. A cosmetic composition according to claim 1, wherein the at least one polyethylene glycol ester is chosen from those of formula (I):
X—(OCH₂CH₂)ₘOY (I)
wherein
m ranges from 80 to 500, and
X and Y, independently from each other, are chosen from a hydrogen atom, and linear and branched, saturated and unsaturated C₈ to C₃₀ acyl groups, with the proviso that at least one of the X and Y groups is an acyl group.

26. A cosmetic composition according to claim 25, wherein the at least one polyethylene glycol ester is a monoester.

27. A cosmetic composition according to claim 25, wherein the at least one polyethylene glycol ester is a diester.

28. A cosmetic composition according to claim 25, wherein m ranges from 100 to 300.

29. A cosmetic composition according to claim 25, wherein the ratio (R), defined as
R=[hydrophilic part weight (OCH₂CH₂)ₘ]/[polyethylene glycol ester weight−hydrophilic part weight (OCH₂CH₂)ₘ]
ranges from 8 to 1,000.

30. A cosmetic composition according to claim 1, wherein the at least one polyethylene glycol ester is present in an amount ranging from 0.01% to 50% by weight relative to the weight of the final cosmetic composition.

31. A cosmetic composition according to claim 30, wherein the at least one polyethylene glycol ester is present in an amount ranging from 0.5% to 15% by weight relative to the weight of the final cosmetic composition.

32. A cosmetic composition according to claim 1, further comprising at least one adjuvant chosen from gelling agents, thickeners, surfactants, silicones, organic solvents, fragrances, mineral oils, vegetable oils, synthetic oils, fatty acid esters, pH stabilizing agents, preserving agents, and UV absorbers.

33. A composition according to claim 1, further comprising at least one propellant and wherein the composition is conditioned in the form of an aerosol.

34. A hair styling method comprising
(a) applying onto the hair a composition comprising in a cosmetically acceptable aqueous medium:
(i) at least one cationic polyurethane comprising at least one non-ionic unit derived from at least one polymer chosen from olefinic homopolymers and copolymers, and
(ii) at least one polyethylene glycol ester;
(b) optionally rinsing the hair, and
(c) styling and drying the hair.
